(19) United States
(12) Patent Application Publication
(10) Pub. No.: US 2008/0033184 A1
(43) Pub. Date: Feb. 7, 2008

(54) INTERMEDIATES AND METHODS FOR SEROTONERGIC AGONIST SYNTHESIS

(75) Inventors: Pete Delgado, Fort Worth, TX (US); W. Dennis Dean, Burleson, TX (US); Raymond E. Conrow, Crowley, TX (US)

Correspondence Address: ALCON, IP LEGAL, TB4-8, 6201 SOUTH FREEWAY, FORT WORTH, TX, 76134, US

(73) Assignee: ALCON MANUFACTURING, LTD., Fort Worth, TX (US)

(21) Appl. No.: 11/832,572

(22) Filed: Aug. 1, 2007

Related U.S. Application Data
(60) Provisional application No. 60/821,102, filed on Aug. 1, 2006.

(51) Int. Cl. C07D 231/56

(52) U.S. Cl. 548/362.5; 548/361.1

(57) ABSTRACT

Disclosed is a method of making a 1-alkylindazole comprising reacting a 1-acylindazole with a first reducing agent, and contacting the resulting mixture with an acid anhydride or acyl halide, and with pyridine or a 4-dialkylaminopyridine or a combination of pyridine and a 4-dialkylaminopyridine, to form a hemiaminal ester and reacting the hemiaminal ester with a second reducing agent to form a 1-alkylindazole. Also disclosed are intermediates for the synthesis of 1-alkylindazoles.

### CROSS-REFERENCE TO RELATED APPLICATION

This application claims priority under 35 U.S.C. §119 to U.S. Provisional Patent Application No. 60/821,102 filed Aug. 1, 2006, the entire contents of which are incorporated herein by reference.

### TECHNICAL FIELD OF THE INVENTION

The present invention is related generally to intermediate compounds and methods for serotonergic agonist synthesis and more specifically to 1-alkylindazole intermediate compounds useful for serotonergic agonist synthesis.

### BACKGROUND OF THE INVENTION

Serotonergic receptor agonists are being investigated as compounds useful for treating a variety of disease states, including the ocular disease glaucoma. The disease state referred to as glaucoma is characterized by a permanent loss of visual function due to irreversible damage to the optic nerve. The several morphologically or functionally distinct types of glaucoma are typically characterized by elevated intraocular pressure (IOP), which is considered to be causally related to the pathological course of the disease. If glaucoma or ocular hypertension is detected early and treated promptly with medications that effectively reduce elevated intraocular pressure, loss of visual function or its progressive deterioration can generally be ameliorated. There is, therefore, a need for therapeutic agents that control IOP.

Certain indazoles are 5-HT serotonergic receptor agonists that have been disclosed as having utility as agents for treating glaucoma and elevated IOP in U.S. Pat. No. 6,696,476 to Chen et al., issued Feb. 24, 2004, the entire contents of which are herein incorporated by reference. It is an object of the present invention to provide additional intermediates and processes for the synthesis of indazoles. Other objects will be evident from the ensuing description and claims.

### SUMMARY OF THE INVENTION

Embodiments of the present invention provide efficient and simplified methods for the synthesis of indazole compounds, particularly indazole compounds that are useful as serotonergic receptor agonists. In particular, embodiments of the present invention provide methods and intermediates for the synthesis of 1-alkylindazoles. Such embodiments comprise a two-step reduction of a 1-acylindazole to give a 1-alkylindazole. In certain embodiments, the 1-alkylindazoles thus formed are useful as or for the synthesis of serotonergic receptor agonists.

In an embodiment, a 1-acylindazole is reacted with a first reducing agent and the resulting mixture is contacted with an acid anhydride or acyl halide to form a hemiaminal ester. The hemiaminal ester is reacted with a second reducing agent to form a 1-alkylindazole. In a preferred embodiment, the first reducing agent is sodium bis(2-methoxyethoxy)aluminum hydride or diisobutylaluminum hydride. Also in a preferred embodiment, the second reducing agent is a trialkylsilane.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. Figures provided herein are intended to help illustrate the invention or assist with developing an understanding of the invention, and are not intended to be definitions of the invention's scope.

### DETAILED DESCRIPTION OF THE INVENTION

Certain 1-alkylindazoles are serotonergic agonists. See U.S. Pat. No. 6,696,476 to Chen et al. 1-Alkylindazoles of the formula QCH₂Y, in which Q is an optionally substituted indazol-1-yl group and Y is an optionally substituted alkyl group, can be prepared by reacting an N-unsubstituted indazole QH with an alkyl halide or other alkylating agent. This method suffers from the disadvantage that the isomeric 2-alkylindazole is formed concurrently in comparable amount. Therefore, material is wasted and a difficult separation must be performed to isolate the desired 1-alkylindazole. See U.S. Pat. No. 6,998,489 to Conrow et al. New methods for the synthesis of 1-alkylindazoles free of their 2-alkylindazole isomers are desirable.

The acylation of an N-unsubstituted indazole QH can give both the 1-acylindazole QC(═O)Y and the isomeric 2-acylindazole. However, in contrast to the foregoing alkylindazole case, the 2-acylindazole can isomerize readily to the 1-acylindazole. This isomerization can be effected by applying heat or certain catalysts, is general for indazoles variously substituted on the carbocyclic ring, and yields the 1-acylindazole free of the 2-acylindazole: Yamazaki et al., Tetrahedron Letters, pg. 4421, 1974. In some cases, this isomerization is found to occur concomitantly with the acylation step: Kingsbury et al., Journal of Medicinal Chemistry, Vol. 19:839, 1976.

It is desirable to have a method for converting 1-acylindazoles to 1-alkylindazoles. Specifically, it is desired to have a method for converting the carbonyl (C═O) group of a 1-acylindazole to a methylene (CH₂) group, thereby producing a 1-alkylindazole. Reaction of a 1-acylindazole with lithium aluminum hydride, which results in the conversion of C═O to CH₂ in N,N-dialkylcarboxamides, does not give the 1-alkylindazole but instead results in undesired carbon-nitrogen bond cleavage. Likewise, reaction of a 1-acylindazole with alane or diborane, as described by Jackson et al., Australian Journal of Chemistry, Vol. 36:779, 1983, for the reduction of azetidine-2-ones, does not give the 1-alkylindazole but instead results in undesired carbon-nitrogen bond cleavage.

In certain embodiments of the present invention, a 1-acylindazole is reacted with a first reducing agent, and the resulting mixture is contacted with an acid anhydride or acyl halide, and with pyridine or a 4-dialkylaminopyridine or a combination of pyridine and a 4-dialkylaminopyridine, to form a hemiaminal ester. In a preferred embodiment, the first reducing agent is sodium bis(2-methoxyethoxy)aluminum hydride or diisobutylaluminum hydride. Also in a preferred embodiment, the resulting mixture is contacted with acetic anhydride and a combination of pyridine and 4-dimethylaminopyridine. The hemiaminal ester formed as above is reacted with a second reducing agent to form a 1-alkylindazole. In a preferred embodiment, the second reducing agent is a combination of a trialkylsilane and boron trifluoride etherate and the trialkylsilane may be, for example, triethylsilane or n-butyldimethylsilane.

The reduction of a hemiaminal ester comprising a 5-acetoxypyrrolidin-2-one with triethylsilane and boron trifluoride etherate is known to effect replacement of the 5-acetoxy group by hydrogen: Hwang et al., Bioorganic and Medicinal Chemistry, Vol. 9:1429, 2001.

The reductive acetylation of carboxylic esters to give α-acetoxy ethers and the reductive deacetoxylation of α-acetoxy ethers are described by Kopecky et al., Journal of Organic Chemistry, Vol. 65:191, 2000.

Specific reaction conditions for the above processes can be readily ascertained by those of skill in the art using the information presented above together with conditions provided below in the Examples.

### EXAMPLES

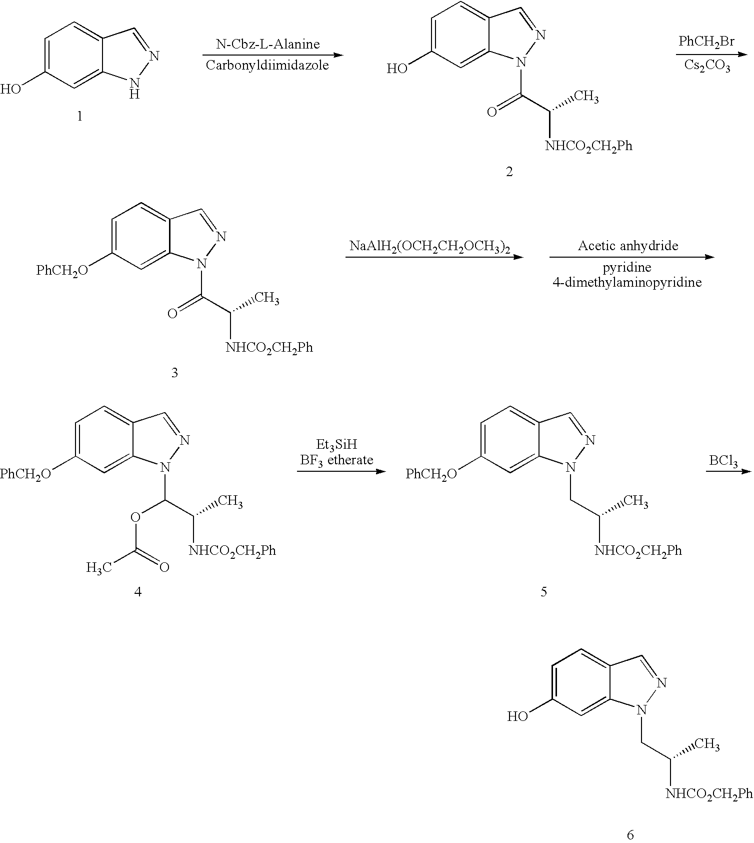

(S)-1-(1-Oxo-2-(benzyloxycarbonyl)amino)propylindazol-6-ol (2). Carbonyldiimidazole (7.04 g, 43.5 mmol) was added to a stirred solution of N-carbobenzyloxy-L-alanine (8.81 g, 39.5 mmol) in 32 mL of dry dimethylformamide under N₂. After CO₂ evolution subsided (15 min), indazol-6-ol (1, 5.29 g, 39.5 mmol) was added. After 18 h, ethyl acetate was added and the mixture was washed with water (3 times) and brine, dried (MgSO₄), filtered and concentrated. The residue was dissolved in 200 mL of 4:1 dichloromethane-ethyl acetate, treated with charcoal and eluted through Florisil and charcoal. The eluate was concentrated to a foam which was crystallized twice from toluene to give 7.80 g (58%) of 2.

(S)-1-(1-Oxo-2-(benzyloxycarbonyl)amino)propyl-6-benzyloxyindazole (3). To a stirred solution of 2 (4.90 g, 14.5 mmol) in 85 mL of dry acetone under N₂ was added benzyl bromide (2.1 mL, 18 mmol) and cesium carbonate (5.6 g, 17 mmol). The mixture was stirred for 15 h, then filtered with the aid of ethyl acetate. The filtrate was washed with water and brine, dried (MgSO₄), filtered and concentrated. The residue was triturated with ice-cold n-BuCl (30 mL) to give 4.33 g (70%) of 3.

1-(1-Acetoxy-2(S)-(benzyloxycarbonyl)amino)propyl-6-benzyloxyindazole (4). A suspension of 3 (3.07 g, 7.16 mmol) in 30 mL of dry toluene and 5 mL of dry dichloromethane was cooled under N₂ to −15° C. A 3.4 M toluene solution of NaAlH₂(OCH₂CH₂OCH₃)₂ (3.4 mL, 11.6 mmol) was added over 15 min. After a further 5 min, acetic anhydride (8.1 mL, 86 mmol) was added, followed by a solution of 4-dimethylaminopyridine (1.05 g, 8.6 mmol) in 15 mL of dry pyridine. The solution was stirred to RT and maintained at RT for 1 h, then cooled in ice and quenched with 1M aqueous NaHSO₄ and ethyl acetate. The layers were separated and the organic solution was washed with water, saturated aqueous NaHCO₃, water and brine, dried (Na₂SO₄), filtered and concentrated to give 3.67 g of 4.

(S)-1-(2-(Benzyloxycarbonyl)amino)propyl-6-benzyloxyindazole (5). A stirred solution of 4 (3.65 g) and triethylsilane (5.6 mL, 35 mmol) in 35 mL of dry dichloromethane under N₂ was cooled to 3-4° C. (bath). Boron trifluoride etherate (4.4 mL, 35 mmol) was added over 2 min. After 10 min, the bath was adjusted to 10° C. After a further 35 min, the mixture was quenched into saturated aqueous NaHCO₃. EtOAc was added and the mixture was stirred until CO₂ evolution ceased. The layers were separated and the organic solution was washed with brine, dried (Na₂SO₄), filtered and concentrated to give 3.09 g of a solid. Recrystallization from 1:1 n-BuC1-hexane gave 1.68 g (57% for 2 steps) of 5.

(S)-1-(2-(Benzyloxycarbonyl)amino)propylindazol-6-ol (6). A stirred solution of 5 (1.61 g, 3.88 mmol) in 25 mL of dry dichloromethane was cooled under N₂ to −45° C. Boron trichloride (14 mL of a 1M solution in dichloromethane) was added in three portions over 30 min. After a further 20 min, the solution was poured into saturated aqueous NaHCO₃. Ethyl acetate was added and the mixture was stirred for 1 h, then separated. The organic solution was washed with brine, dried (MgSO₄), filtered and concentrated to a gum, which was triturated with hot toluene. The toluene solution was treated with charcoal, filtered and concentrated. The product was crystallized from n-BuCl to give 1.00 g (79%) of 6.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

We claim:

1. A compound of the formula: 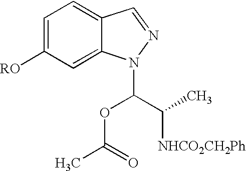
wherein R═H, CH₃OCH₂, CH₃CH₂OCH(CH₃), PhCH₂ or 4-CH₃OC₆H₄CH₂.

2. The compound of claim 1 wherein R=PhCH₂.

3. A method of making a 1-alkylindazole comprising:
reacting a 1-acylindazole with a first reducing agent, and contacting the resulting mixture with an acid anhydride or acyl halide, and with pyridine or a 4-dialkylaminopyridine or a combination of pyridine and a 4-dialkylaminopyridine, to form a hemiaminal ester; and
reacting said hemiaminal ester with a second reducing agent to form a 1-alkylindazole.

4. The method of claim 3 wherein said 1-acylindazole is (S)-1-(1-oxo-2-(benzyloxycarbonyl)amino)propyl-6-benzyloxyindazole.

5. The method of claim 3 wherein said 1-alkylindazole is (S)-1-(2-(benzyloxycarbonyl)amino)propyl-6-benzyloxyindazole.

6. The method of claim 3 wherein said first reducing agent is sodium bis(2-methoxyethoxy)aluminum hydride or diisobutylaluminum hydride.

7. The method of claim 3 wherein the acid anhydride is acetic anhydride.

8. The method of claim 3 wherein said mixture is contacted with a combination of pyridine and a 4-dialkylaminopyridine, and wherein said 4-dialkylaminopyridine is 4-dimethylaminopyridine.

9. The method of claim 3 wherein said second reducing agent is a combination of a trialkylsilane and boron trifluoride etherate.

10. The method of claim 9 wherein said trialkylsilane is triethylsilane or n-butyldimethylsilane.

11. A method of making (S)-1-(2-(benzyloxycarbonyl)amino)propyl-6-benzyloxyindazole comprising:
reacting (S)-1-(1-oxo-2-(benzyloxycarbonyl)amino)propyl-6-benzyloxyindazole with sodium bis(2-methoxyethoxy)aluminum hydride, and contacting the resulting mixture with acetic anhydride and a combination of pyridine and 4-dimethylaminopyridine, to form a hemiaminal ester; and
reacting said hemiaminal ester with a combination of triethylsilane and boron trifluoride etherate to form (S)-1-(2-(benzyloxycarbonyl)amino)propyl-6-benzyloxyindazole.
